(19) United States
(12) Patent Application Publication
(10) Pub. No.: US 2010/0049284 A1
(43) Pub. Date: Feb. 25, 2010

(54) USE OF HEAT TO TREAT BIOLOGICAL SYSTEMS EXPOSED TO DAMAGING RADIATION

(75) Inventors: PATRICK M. MEHL, Cabin John, MD (US); James K. Keeven, Chantilly, VA (US)

Correspondence Address: Vedder Price, PC, 875 15th Street, NW, Suite 725, Washington, DC, 20005, US

(73) Assignee: The Catholic University of America, Washington, DC (US)

(21) Appl. No.: 12/342,975

(22) Filed: Dec. 23, 2008

Related U.S. Application Data
(60) Provisional application No. 61/053,313, filed on May 15, 2008.

(51) Int. Cl. A61F 7/12

(52) U.S. Cl. 607/100; 607/96; 607/105; 607/103; 601/3

(57) ABSTRACT

A method wherein, following a therapeutic radiation treatment, a heat dose is administered to the treated cells to decrease the damage caused by radiation exposure or a therapeutic agent.

### PRIORITY DATA AND INCORPORATION BY REFERENCE

This application claims benefit of priority to U.S. Provisional Patent Application No. 61/053,313 entitled “Use of Heat to Treat Biological Systems Exposed to Therapeutic Radiation” filed May 15, 2008 which is incorporated by reference in its entirety.

### BACKGROUND OF THE INVENTION

1. Field of the Invention

The present invention relates to a method for treating biological systems exposed to damaging radiation.

2. Background of the Technology

There are deficiencies in all of the treatments currently available for mitigating the damaging effects of radiation exposure.

### SUMMARY OF THE INVENTION

According to a first broad aspect, the present invention provides a method comprising the following steps: (a) providing cells exposed to damaging radiation; and (b) administering a heat dose that is sufficient to induce a measurable stress response in the cells, wherein the heat dose is administered to the cells at a temperature of between about 39° C. and about 45° C. for at least about 5 minutes and wherein the heat dose is administered to the treated cells within about 240 minutes after the cells are exposed to the damaging radiation.

According to a second broad aspect, the present invention provides a method comprising the following steps: (a) providing cells treated with a therapeutic agent; and (b) administering a heat dose that is sufficient to induce a measurable stress response in the cells, wherein the heat dose is administered to the cells at a temperature of between about 39° C. and about 45° C. for at least about 5 minutes and wherein the heat dose is administered to the treated cells within about 240 minutes after the cells are treated with the therapeutic agent.

### BRIEF DESCRIPTION OF THE DRAWINGS

The accompanying drawings, which are incorporated herein and constitute part of this specification, illustrate exemplary embodiments of the invention, and, together with the general description given above and the detailed description given below, serve to explain the features of the invention.

FIG. 1 is a graph showing mouse fibroblast L929 survival determined using a colony forming assay after exposure to 3 Gy of X-ray radiation, with and without a heat treatment after the X-ray exposure; L929 are muscle cell type;

FIG. 2 is a graph showing the survival of human endothelial cadmec cells exposed to 3 Gy of X-ray radiation, followed by no heat treatment after X-ray exposure, or treatment with a heat dose of 41.5° C. after the X-ray exposure, compared to a control; and these cells are micro vasculature cell type; and

FIG. 3 is a graph showing H₂O₂ degradation rate compared to a control in mouse fibroblast L929 cells treated with ultraviolet subtype C (UVC), heat treatment alone, or UVC followed by a heat treatment.

### DETAILED DESCRIPTION

It is advantageous to define several terms before describing the invention. It should be appreciated that the following definitions are used throughout this application.

### Definitions

Where the definition of terms departs from the commonly used meaning of the term, applicant intends to utilize the definitions provided below, unless specifically indicated.

For the purposes of the present invention, directional terms such as “top”, “bottom”, “upper”, “lower”, “above”, “below”, “left”, “right”, “horizontal”, “vertical”, “upward”, “downward”, etc., are merely used for convenience in describing the various embodiments of the present invention.

For the purposes of the present invention, the term “accidental radiation exposure” refers to an unintentional exposure to radiation. Examples of accidental radiation exposure include: exposure to radiation leaks, spatters, discharges, seepages, or spills, nuclear fallout, nuclear reactor accidents, background radiation, production, use, disposal, etc., of radioactive materials, for example, use in nuclear medicine, disposal of radioactive waste, etc., plants, animals, drinking water, etc., contaminated with radioactive materials, etc.

For the purposes of the present invention, the term “cadmec” refers to cadaveric micro endothelial cells from humans that are essential for the micro-vascularization of tissues.

For the purposes of the present invention, the term “cancer” refers to any type of cancer including, but not limited to, skin cancer, breast cancer, lung cancer, pancreatic cancer, colorectal cancer, prostate cancer, bladder cancer, endometrial cancer, ovarian cancer, leukemia, lymphomas, etc.

For the purposes of the present invention, the term “cells” refers to cells either in vivo or in vitro. Cells may be part of a tissue culture, present in an individual, present in an animal, etc. Cells may also include bacteria, viruses, fungi, archaea, prions, etc.

For the purposes of the present invention, the term “chemotherapeutic agents” refers to any chemotherapeutic or cytotoxic agent including, but not limited to, taxanes such as paclitaxel (taxol) and docetaxel, alkylating agents such as cisplatin, carboplatin, oxaliplatin, mechlorethamine, cyclophosphamide, and chlorambucil, antimetabolites such as azathioprine, mercaptopurine, vinca alkaloids such as vincristine, vinblastine, vinorelbine, and vindesine, a podophyllotoxin-derived toxin such as etoposide and teniposide, topoisomerase inhibitors such as irinotecan and topotecan (type 1) and amsacrine (type 2), antitumour antibiotics such as dactinomycin, doxorubicin, epirubicin, and bleomycin, monoclonal antibodies such as trastuzumab (Herceptin), cetuximab, and rituximab (Rituxan or Mabthera), and evacizumab (Avastin), etc.

For the purposes of the present invention, the term “chemotherapeutic treatment” or “chemotherapy” refers to treatment of an individual with a chemotherapeutic agent such as in chemotherapy treatments for cancer.

For the purposes of the present invention, the term “constitutive levels” refers to the levels (concentrations) of heat shock proteins in a cell prior to a treatment with low dose radiation (LDR) or other stress in accordance with embodiments of the present invention, i.e., the normal levels of heat shock proteins in the cells.

For the purposes of the present invention, the term “heat dose” refers to exposing cells or tissues to a quantity of heat at a particular temperature or within a particular temperature range for a period of time.

For the purposes of the present invention, the term “heat shock protein” or “Hsp” refers to any stress induced protective molecules that are induced by a variety of environmental stresses such as heat, ionizing and non-ionizing radiation (including electromagnetic fields and time varying magnetic fields), toxic chemicals, hypoxia (low oxygen), incorrect glucose levels, heavy metals and amino acid analogs, etc. An important protective cellular mechanism involves activation of a stress response related to the induction of heat shock proteins (Hsps). Hsps are often named according to their molecular weight. For example, Hsp60, Hsp70 and Hsp90 refer to Hsp families on the order of 60, 70 and 90 kilodaltons in size. The Hsp family may comprise proteins synthesized in response to oxidative stress, which may then provide cellular protection from subsequent damage. Hsp70/72 is the most widely inducible protein in this family. See, for example, heat induction of Hsps in lymphocytes prior to radiation delayed apoptosis (see Gordon et al., Arch. Surg., 132 (12):1277-1282 (1997)), and Hsp over-expression inhibited cell death following lethal x-ray exposure (see Park et al., Radiat. Res. 153(3):318-326 (2000)). LDR exposure has been shown to enhance Hsp levels (see Nogami et al., Int. J. Radiat. Biol., 63(6): 775-783 (1993); and Melkonyan et al., Int. J. Rad. Biol., 68(3):277-280 (1995). Sato et al. also shows that low dose X-ray irradiation induces Hsps in the gastric mucosa (see Physiol. Phys. Chem. & Med. NMR, 28:103-109 (1996)), and others (see O'Rourke et al., Biochem. Soc. Trans., 20(1):74S (1992)) have presented evidence that LDR exposure induces Hsps in cultured myeloid leukemia and CHO cells. The entire contents and disclosures of the above documents are incorporated herein by reference.

For the purposes of the present invention, the term “high dose radiation” and “HDR” refers to any dose over 0.5 Gy or any dose that might be used therapeutically to kill cells.

For the purposes of the present invention, the term “individual” refers to a mammal such as a human being, monkey, chimpanzee, horse, dog, cat, rodent (e.g., mouse), hamster, etc.

For the purposes of the present invention, the term “ionizing radiation” refers to ionizing radiation from any source that is capable of ionizing atoms, molecules, etc., of a cell. Examples of ionizing radiation include: X-rays, ultraviolet light, gamma rays, alpha particles, beta particles, neutrons, etc. Depending upon the wavelength, ultraviolet light may be considered either ionizing or non-ionizing radiation.

For the purposes of the present invention, the term “lethal dose” refers to a dose of radiation or any other therapeutic agent or modality that is capable of killing, or modifying the function of, one or more cells. A commonly-used lethality indicator is the LD₅₀ (or LD50), a dose at which 50% of subject individuals will normally die.

For the purposes of the present invention, the term “low dose radiation (LDR)” refers to a dose of ionizing radiation in the range of from about 0.5 to about 50 cGy. In one embodiment of the present invention, an LDR dose of from about 1 to about 10 cGy may be used to LDR-sensitize cancer cells. Other dosages of LDR may be used in other embodiments of the present invention depending on the type of tissue to be treated, the disease to be treated, etc.

For the purposes of the present invention, the terms “measurable increase in the concentration of a stress protein” or “measurable stress response” refer to at least about a 7% increase in the concentration of a stress protein in a treated tissue after being exposed to a heat dose compared to the concentration of the stress protein in the treated tissue prior to being exposed to the heat dose.

For the purposes of the present invention, the term “modifies the functioning of a cell” refers to any alteration in the normal functioning of a cell. Examples of modifications include such modifications as: changing the proliferation rate of cells, etc.

For the purposes of the present invention, the term “pharmaceutical mitigator” refers to one or more chemical compounds administered to a tissue or individual to mitigate the effects of radiation damage. Pharmaceutical mitigators, such as vitamin E or amifostine, may be applied topically to the tissue, ingested by the individual, or administered in any other appropriate way for the particular pharmaceutical mitigator. Depending on the particular pharmaceutical mitigator, the pharmaceutical mitigator may be administered to the tissue being treated with radiation or to the individual whose tissue is being treated with radiation before the radiation treatment, during the radiation treatment, or after the radiation treatment. If the pharmaceutical mitigator is administered after the radiation treatment, depending on the type of pharmaceutical mitigator, the pharmaceutical mitigator may be administered before, during or after the heat dose is applied to the treated tissue following the radiation treatment. Depending on the mode of action of the pharmaceutical mitigators (such as vitamin E protecting the cell membranes, free radical scavenger agents that can decrease the presence of free radicals that are early responders, or other mitigators activating rescue processes activating DNA protection and repairs that are late responders), heat ,may be applied at different timings. The pharmaceutical mitigator may be administered orally, topically, intravenously, parenterally, intramuscularly, subcutaneously, intranasally, transdermally, as a rectal suppository, etc. Suitable pharmaceutical mitigators may include vitamin E (tocopherols and tocotrienols) and its derivatives, other antioxidants and antioxidant nutrients such as selenium, zinc, vitamin A, vitamin C, anti-apoptotic agents such as co-enzyme Q10 (also known as ubiquinone or ubidecarenone), ursidiol (ursodeoxycholic acid) or apoptosis inhibitors, radioprotective agents (radioprotectants) such as amifostine, etc. Representative anti-apoptotic agents may include chemokines, dipeptide apoptosis inhibitors, anti-apoptotic proteins such as Bcl-2, Bcl-X_{L} mutant p53, caspase inhibitors such as caspase-9-DN and caspase-9S. baculovirus p35, DADI or AAC-11 proteins, 1-heteroazolyl-1-heterocyclyl)alkane derivatives, 3 S,4aR,6R, 8aR-6-[2-(1 H-tetrazol-5-yl)-ethyl-1,2,3,4,4a,5,6,7,8,8a-decahydroisoquinoline-3-carboxylic acid, etc. See, for example, U.S. Pat. No. 5,824,551 (Damme et al.), issued Oct. 20, 1998; U.S. Pat. No. 6,184,210 (Keana et al.), issued Feb. 6, 2001; U.S. Pat. No. 6,949,516 (Keana et al.), issued Sep. 27, 2005; U.S. Pat. No. 7,071,382 (Cahoon et al.), issued Jul. 4, 2006; U.S. Pat. No. 6,586,206 (Dixit et al.), issued Jul. 1, 2003; and U.S. Pat. No. 6,555,565 (Matsui et al.), issued Apr. 29, 2003, the entire contents and disclosures of which are herein incorporated by reference. Representative radioprotectants may include aminoalkyl phosphorothioate and/or aminoalkyl thiol compounds, for example, S-2-(3-aminopropylamino) ethyl phosphophorothioic acid (also known as WR-2721), a thiophosphate derivative of aminothiol cysteamine and 2-(3-aminopropyl) aminoethanethiol (amifostine, also known as WR-1065), α,β-unsaturated arylsulfones, etc. See, for example, U.S. Pat. No. 5,434,145 (Edwards et al.), issued Jul. 18, 1995); U.S. Pat. No. 6,114,394 (Edwards et al.), issued Sep. 5, 2000; U.S. Pat. No. 6,489,312 (Stogniew et al.), issued Dec. 3, 2002; U.S. Pat. No. 6,573,253 (Stogniew), issued Jun. 30, 2003; U.S. Pat. No. 6,667,346 (Reddy et al.), issued Dec. 23, 2002; and U.S. Pat. No. 7,368,440 (Cassatt et al.), issued May 6, 2008, the entire contents and disclosures of which are herein incorporated by reference.

For the purposes of the present invention, the term “radiation attack” refers to radiation generated or released for the purpose, objective, intention, etc., of harming, damaging, hurting, killing, etc., one or more individuals. Examples of radiation attacks may include: attacks made by terrorists using radiation, such as by a nuclear weapon, a radioactive dispersal device, (e.g., a “dirty bomb”); attacks by terrorists causing radiation to be released, dispersed, etc., such as by damaging a nuclear reactor; attempting to harm or assassinate individuals by exposing the individuals to radiation by, for example, injecting, poisoning, etc., individuals with radioactive materials, etc.

For the purposes of the present invention, the term “stress inducing agent” refers to agents that induce a stress response. A stress inducing agent may be radiation-based, chemically-based, physically-based, heat-based, etc.

For the purposes of the present invention, the term “therapeutic agent” refers to any agent or procedure such as radiation, chemicals used in chemotherapy, pharmaceuticals, etc., that may be used to reduce the number of or to prevent the proliferation of the cells that are the target of a therapeutic treatment.

For the purposes of the present invention, the term “therapeutic treatment” refers to the administering of a therapeutic agent to an individual in a dosage or manner sufficient to reduce the number of one or more types of cells or to prevent the proliferation of one or more type of cells.

For the purposes of the present invention, the term “therapeutically effective amount” refers to an amount of a therapeutic agent capable of inhibiting the functioning or proliferation of cells and or reducing the number of living cells.

For the purposes of the present invention, the term “treated cells” refers to cells treated with a therapeutic agent or cells in an individual treated with a therapeutic agent. An individual and/or the cells of an individual may be treated with therapeutic agent in a various ways including: orally, topically, intravenously, parenterally, intramuscularly, subcutaneously, intranasally, transdermally, as a rectal suppository, etc. The treated cells may be a biological organism, such as bacteria or a virus, may be cells of a tissue or may be an entire tissue.

For the purposes of the present invention, the term “treated tissue” refers to tissue treated with a therapeutic agent or tissue in an individual treated with a therapeutic agent. An individual and/or the tissue of an individual may be treated with therapeutic agent in a various ways including: orally, topically, intravenously, parenterally, intramuscularly, subcutaneously, intranasally, transdermally, as a rectal suppository, etc. The treated tissue may be separate tissue, part of an organ or an entire organ.

For the purposes of the present invention, the term “tumor” refers to any type of tumor including both malignant and non-malignant tumors and any abnormal proliferation of cells.

### Description

It is known that heat treatment before radiation exposure may provide some protection to biological systems from the damaging effects of that radiation. In fact, heat treatments have been used for a long time for treatment of a variety of conditions from cancer therapies where high heat is used to facilitate cancer cell killing, to muscle sprains where heat is used for pain relief But the use of a heat dose to rescue biological systems from damage after radiation exposure has not previously been considered. Apparently, the ability to mitigate the damage of radiation exposure by a subsequent heat treatment is considered by those skilled in the art to be unexpected and surprising.

In one embodiment, the present invention involves the use of a heat dose after radiation exposure to enhance survival. The heat dose used in embodiments of the present invention may be administered in a variety of ways. For example, the heat dose may be administered by direct contact of the treated cells with a heated surface, by direct contact of the treated cells with a heated liquid, by exposing the treated cells to infrared radiation, by exposing the treated cells to ultrasound, etc.

The levels and duration of heating that may be employed by various embodiments of the present invention may be easily produced without requiring complex equipment. Also, heat doses at the temperatures employed by various embodiments of the present invention should desirably not cause any near or long term side effects, toxicities, etc.

In one embodiment, heat doses after radiation exposure maybe used in combination with other potential pharmaceutical mitigators of radiation damage such as vitamin E (tocopherols and tocotrienols) and its derivatives, other antioxidants or antioxidant nutrients such as vitamin A, vitamin C, selenium, zinc, anti-apoptotic agents, radioprotective agents (radioprotectants), etc.

In one embodiment, the method may be used to promote wound healing after trauma or surgical procedures or protect heart tissue from the damaging effects of reoxegenation and reactive oxygen species production after an individual suffers a heart attack. Pretreatment with heat for surgery in a rodent has been successfully used in protecting the heart tissue against the presence of reactive oxygen species produced during the transplantation and reperfusion (A. Gowda et al., Ann Thorac Surg, 66:1991-1997 (1998).

In one embodiment, the heat treatment is applied within about 240 minutes after radiation treatment. In one embodiment, the heat treatment is applied within from about 1 to about 120 minutes after the radiation treatment for treated cells at a temperature below 39° C. In one embodiment, the heat treatment is applied within from about 5 to about 60 minutes after the radiation treatment to allow the temperature of the cells to reach a temperature below about 38.5° C. before applying embodiments of the present therapeutic heat treatment.

In order to provide heat treatment shortly after radiation treatment, the following techniques may be employed, including direct heat application techniques such as direct contact with a heat bath within a container such a deformable heated flask, a hat having heating elements or a hot or heated liquid contained therein (e.g., within a wall or chamber of the hat, tubing within or surrounding the hat, etc.) for heating the head, gloves, mittens, foot coverings (e.g., shoes, slippers, etc.) or other clothes (e.g. shirts, pants, slacks, scarves, robes, gown, coats, etc.) with heating elements or heated liquid contained therein (e.g. within in a wall or chamber of the clothes, tubing in the hat, etc.), a deformable heating (electric) blanket, cover, bedspread, etc., or direct radiation such as with infra-red or near infra-red lamps for deeper penetration, LED networks, magnetic nanoparticles that can be perfused within the patient (e.g., using magnetohypothermia techniques).

The time and temperature of treatment may be dependent on the heat susceptibility of the biological system. In one embodiment, the temperature of the heat dose administered may be in the range of from about 39° to about 45° C. depending on the exposure time. In one embodiment, the heat dose may be administered at a temperature above about 39° C. to be capable of inducing a cellular response, but below about 43° C. to avoid the induction of an apoptotic response as may be seen with hyperthermia treatment of cancer. In one embodiment, the heat dose may be administered between about 39° C. and about 42° C. for sensitive tissues.

In one embodiment, the duration of the heat treatment may be between about 1 minute and about 120 minutes. In one embodiment, the duration of the heat treatment may be between about 5 minutes and about 60 minutes. In one embodiment, the duration of the heat treatment may be between about 5 minutes and about 30 minutes. In one embodiment, the duration of the heat treatment may be between about 10 minutes and about 30 minutes.

The means of applying the heat treatment may be different depending upon the biological systems and type of radiation exposure. In one embodiment of the present invention, direct heat transfer by conduction may be used to provide a heat dose to the treated tissue. For example, the treated tissue may be contacted with a heated surface such as a heated metal, plastic or rubber surface. Examples suitable heated surfaces that may be used to administer a heat dose of the present invention include: liquid thermal containers such as bottles, bags, clothes, water/liquid mattresses, etc. In other embodiments, direct heat transfer by conduction may be performed by contacting the treat tissue with a heated and sterilized liquid such as water, oil for total body exposure such as hot baths with hot liquids, such as water, salt water, mud baths, organic baths, or combination or combinations thereof, by contacting the treat tissue with a heating element such as a heated (electric) blanket, heating pad, etc.

In one embodiment of the present invention, convection may be used to provide a heat dose to the treated tissue. A convection heat dose may be useful for penetrating surface tissues such as skin up to few centimeters. Suitable convection systems that may be used to administer a heat dose that provides a total body exposure, include: sauna rooms or hot air rooms, for local body exposure, directional blowing hot air fans, etc.

In one embodiment of the present invention, direct radiation such as infrared or near infrared radiation may be used to provide a heat dose to the treated tissue. An infrared or near infrared heat dose may be useful for penetrating surface tissues such as skin up to about 2 cm depending on the wavelength and power of the heater. Examples of suitable heating systems employing infrared or near infrared radiation that may be used in the present invention are described in heaters with ceramic infra-red emitters or metal or quartz tubes. The infrared heat dose may have a wavelength between about 2800 nm and about 10000 nm. The near infrared heat dose may have a wavelength between about 700 nm and about 2800 nm.

In one embodiment of the present invention, magnetic or metallic particles directed to internal sites may be used to focus the heating internally. These particles may then be used to heat their surroundings using external magnetic fields (e.g. by magnetohypothermia). An example of such a magnetic particle heating system is described in the treatment of breast cancer using hyperthermia induced by magnetic nanoparticles within a radiofrequency magnetic field (Hilger et al. Nanobiotechnology, IEE proceedings, 152:33 (2005)). See also Pavon et al., “Applications of Nanobiotechnology in Cancer,” Einstein, 5(1):74-77 (2007) for the use of magnetohypothermia in treating cancer.

Embodiments of the heat treatment of the present invention may be used concurrently with potential pharmaceutical mitigators of radiation damages such vitamin E (tocopherols and tocotrienols) and its derivatives, other antioxidants and antioxidant nutrients, anti-apoptotic agents or apoptosis inhibitors, radioprotective agents (radioprotectants), etc.

One benefit of heat treatment according to embodiments of the present invention is providing a therapeutic “stress” that causes cells to synthesize the family of proteins known as HSPs. These HSPs help the cell survive by assisting damaged cells with repair and protecting them against further harm or damage. The increase in the concentration of stress proteins may be measured (and monitored) in a variety of ways. For example, the increase in concentration of stress proteins may be measured by immunoblotting assays.

In addition to administering heat doses to mitigate the effects of therapeutic radiation treatment, embodiments of the method of the present invention may also be used to mitigate the effects of radiation on individuals who have been exposed to radiation accidentally. For example, a heat dose may be administered to an individual at a nuclear plant who has been accidentally exposed to a dangerous amount of radiation.

Embodiments of the method of the present invention may also be used to mitigate the effects of radiation on individuals who have been exposed to radiation due to a radiation attack by a terrorist, an assassin, etc.

Embodiments of the method of the present invention also envision that administering a heat dose may be used in mitigating the effects of therapeutic agents such as chemotherapeutic agents.

### EXAMPLES

### Example 1

Experiments using biochemical assays of cell survival such as proliferation assays using DNA levels and esterase metabolic assays are developed in the lab with mouse fibroblast L929 cells to determine the effect of treating cells with a heat dose after the cells have been exposed to x-ray radiation exposure. Quantitative determination of numbers of surviving cells involve waiting 10 to 21 days for surviving cells to form visible colonies in the background of dead cells. In brief these quantitative experiments involve the use of four sets of flasks with L929 cells:
(1) flasks where cells are not exposed to any treatment;
(2) flasks where cells are exposed only to heat to determine the effect of the heat treatment alone, the heat treatment being an exposure to a temperature of 41.5° C. for 15 minutes;
(3) flasks where cells are exposed to a high dose radiation (HDR) of 3 Gy of x-ray radiation only; and
(4) flasks where cells are exposed to 3 Gy of X-ray radiation (HDR) followed by 30 min rest and then heat treatment.

After treatment cells are collected and dilutions plated in six well plates for long term survival studies. These cultures are visually checked for colony formation after a period of 10 to 21 days. Surviving cells form colonies which are stained and counted.

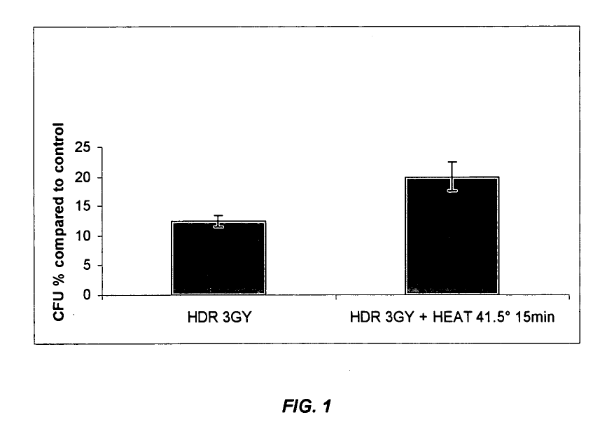

Survival is expressed as the ratio of the average colony number of the treated samples to the average colony number of the control untreated samples. This ratio is expressed as a survival percentage compared to the untreated control as shown in FIG. 1. The use of a non damaging heat treatment after x-ray exposure may increase survival by up to 100%. Heat alone (without X-ray exposure) does not affect survival (data not shown).

Levels of radiation that kill 90% of the L929 cells, as used for these studies, are much higher than the lethal dose for a person. Increasing survival by up to 100% under conditions where 50% of cells are killed (conditions that would still normally be lethal for a person) may lead to close to 100% survival of cells and the difference between life and death for the exposed person.

### Example 2

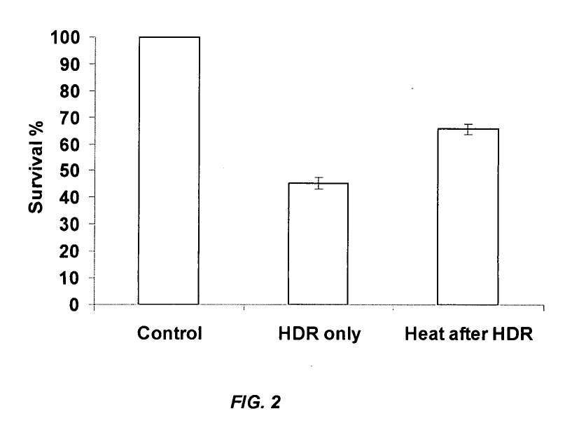

Microvascular endothelial cadmec cells are also tested. Cells exposed to a 3 Gy lethal X-ray radiation dose are rescued with a heat treatment of 15 min at 41.5° C. within 10 minutes after the X-ray radiation (see FIG. 2). As shown in FIG. 2, survival may increase from about 45% to about 75% after heat treatment.

### Example 3

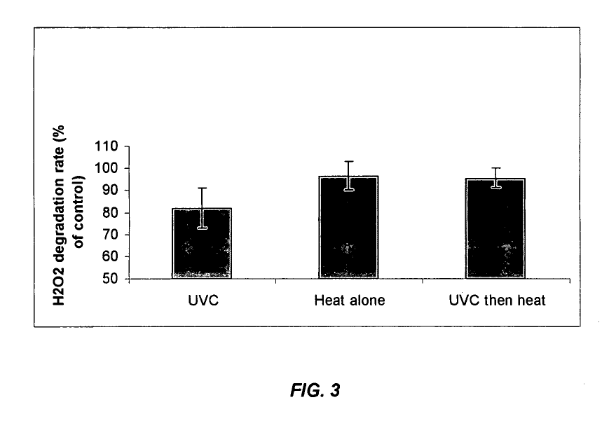

FIG. 3 illustrates that a heat dose at a temperature of 41.5° C. for 15 minutes that is first administered within 5 minutes after exposure to levels of ultraviolet radiation (UV) that would kill 86% of the cells leads to levels of hydrogen peroxide degradation similar to levels seen with heat alone and greater after 1 hour than levels in cells treated with UV alone. Hydrogen peroxide degradation rate is the rate at which the cell is capable to eliminate hydrogen peroxide by biochemical transformation. Hydrogen peroxide is the stable oxidant chemical entity that is formed during an oxidative stress such as radiation. The determination of the hydrogen peroxide degradation rate is therefore measuring the level of plasma membrane biochemical integrity after treatment. This shows that heat treatment after radiation exposure may elicit the same, if not a better, protective response against reactive oxygen species than heat treatment alone and provides one possible mechanism for the observed protection.

While the present invention has been disclosed with references to certain embodiments, numerous modification, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. A method comprising the following steps:
(a) providing cells exposed to damaging radiation; and
(b) administering a heat dose that is sufficient to induce a measurable stress response in the cells, wherein the heat dose is administered to the cells at a temperature of between about 39° C. and about 45° C. for at least about 5 minutes and wherein the heat dose is administered to the treated cells within about 240 minutes after the cells are exposed to the damaging radiation.

2. The method of claim 1, wherein the exposed cells comprise a tissue.

3. The method of claim 1, wherein the exposed cells comprise an organ.

4. The method of claim 1, wherein the cells are exposed to the damaging radiation during an accidental exposure to the damaging radiation.

5. The method of claim 1, wherein the cells are exposed to the damaging radiation during a radiation attack.

6. The method of claim 1, wherein the cells are exposed to the damaging radiation by exposing the treated cells to a therapeutic dose of radiation.

7. The method of claim 1, wherein the heat dose is administered after the exposed cells have reached a temperature of no greater than about 38.5° C.

8. The method of claim 1, wherein the heat dose is administered within from about 1 to about 120 minutes of the end of the exposure of the cells to the damaging radiation.

9. The method of claim 1, wherein the heat dose is administered within from about 5 to about 60 minutes of the end of the exposure of the cells to the damaging radiation.

10. The method of claim 1, wherein the heat dose is administered to the exposed cells at a temperature of between about 39° C. and about 47° C.

11. The method of claim 1, wherein the heat dose is administered to the exposed cells at a temperature of between about 39° C. and about 45° C.

12. The method of claim 1, wherein the heat dose is administered to the exposed cells at a temperature of between about 39° C. and about 43° C.

13. The method of claim 1, wherein the heat dose is administered to the exposed cells for between about 1 minutes and about 120 minutes.

14. The method of claim 1, wherein the heat dose is administered to the exposed cells for between about 5 minutes and about 60 minutes.

15. The method of claim 1, wherein the heat dose is administered to the exposed cells for between about 10 minutes and about 30 minutes.

16. The method of to claim 1, wherein the heat dose is administered to the exposed cells by direct contact of the exposed cells with a heated surface.

17. The method of to claim 1, wherein the heat dose is administered to the exposed cells by direct contact of the exposed cells with a heated liquid.

18. The method of to claim 1, wherein the heat dose is administered to the exposed cells by heating the exposed cells with infrared radiation.

19. The method of to claim 1, wherein the heat dose is administered to the exposed cells by heating the treated cells with ultrasound.

20. The method of claim 1, wherein the heat dose is administered to the exposed cells by heating metallic particles that are in and/or adjacent to the exposed cells with a magnetic field.

21. The method of claim 1, wherein the exposed cells are part of an individual and wherein the method further comprises the following step:
(c) administering a pharmaceutical mitigator to the individual.

22. A method comprising the following steps:
(a) providing cells treated with a therapeutic agent; and
(b) administering a heat dose that is sufficient to induce a measurable stress response in the cells, wherein the heat dose is administered to the cells at a temperature of between about 39° C. and about 45° C. for at least about 5 minutes and wherein the heat dose is administered to the treated cells within about 240 minutes after the cells are treated with the therapeutic agent.

23. The method of claim 1, wherein the therapeutic agent comprises one or more chemotherapeutic agents.
